(19) United States
(12) Patent Application Publication
(10) Pub. No.: US 2015/0087065 A1
(43) Pub. Date: Mar. 26, 2015

(54) METHOD FOR GENERATION OF CONDITIONALLY IMMORTALIZED HEMATOPOIETIC PROGENITOR CELL LINES WITH MULTIPLE LINEAGE POTENTIAL

(71) Applicant: St. Jude Children's Research Hospital, Memphis, TN (US)

(72) Inventor: Hans Haecker, Memphis, TN (US)

(73) Assignee: St. Jude Children's Research Hospital

(21) Appl. No.: 14/394,136

(22) PCT Filed: Apr. 18, 2013
(86) PCT No.: PCT/US2013/037066

§ 371 Date: Oct. 13, 2014

Related U.S. Application Data
(60) Provisional application No. 61/636,080, filed on Apr. 20, 2012.

(51) Int. Cl. C12N 5/0789; C12N 5/0784; C12N 5/0786; C12N 5/0783; C12N 5/0781; C12N 5/078; C12N 5/0787

(52) U.S. Cl. CPC C12N 5/0647; C12N 5/0644; C12N 5/0639; C12N 5/0642; C12N 5/0636; C12N 5/0635; C12N 5/0645; C12N 2506/21; C12N 2506/03; C12N 2501/60; C12N 2501/10; C12N 2501/30; C12N 2501/22; C12N 2502/21
USPC 435/455; 435/377; 435/325; 435/355

(57) ABSTRACT

The present invention is a method and kits for generating a homogenous population of hematopoietic progenitor cells capable of differentiating into a hematopoietic cell lineage. Whereas the combination of Homeobox-B8 protein and FMS-like tyrosine kinase 3 ligand generate cells with the potential to differentiate into different myeloid and lymphoid cell types, Homeobox-A7 protein and erythropoietin generate cells with the potential to differentiate into erythropoietic or thrombopoietic cell types.

### BACKGROUND OF THE INVENTION

The evolutionary conserved, clustered family of Hox genes encodes 39 DNA-binding transcription factors in mammals which control many aspects of embryonic development and hematopoiesis (He, et al. (2011) Oncogene 30:379-388; Argiropoulos & Humphries (2007) Oncogene 26:6766-6776). In one approach using hematopoiesis, Hox genes are preferentially expressed in immature progenitor cells and hematopoietic stems cells (HSC), and are downregulated during cell differentiation and maturation (Giampaolo, et al. (1994) Blood 84:3637-3647; Sauvageau, et al. (1994) Proc. Natl. Acad. Sci. USA 91:12223-12227; Giampaolo, et al. (1995) Stem cells 13 Suppl 1:90-105; Kawagoe, et al. (1999) Leukemia 13:687-698; Pineault, et al. (2002) Exp. Hematol. 30:49-57; Morgan & Whiting (2008) Internatl. J. Hematol. 87:246-249). Based on mouse knockout and overexpression studies, as well as observations derived from oncogenic fusion proteins involving Hox genes themselves or their activators, it has been suggested that one important Hox gene-encoded function is the regulation of cell differentiation, specifically an increase in cell self-renewal and arrest in cell differentiation (Argiropoulos & Humphries (2007) supra). This property has also been used experimentally to establish stably growing, homogenous hematopoietic progenitor cells lines through retroviral-mediated expression of certain Hox genes, such as Hoxa9 and Hoxb8 (Wang, et al. (2006) Nature Meth. 3:287-293; US 2009/0068157; U.S. Pat. No. 6,884,589). In estrogen-regulated forms of Hoxb8 and Hoxa9, wherein the Hox coding sequence is fused to the hormone binding domain of the estrogen receptor (ERHBD), it has been shown that activation of these Hox genes in the presence of estrogen and the addition of the growth factors SCF (Stem Cell Factor) or GM-CSF (Granulocyte-Macrophage Colony-Stimulating Factor) leads to conditional immortalization of committed myeloid progenitor cells of the granulocyte and monocyte lineage, respectively (Wang, et al. (2006) supra). This system not only provides a valuable experimental tool for investigation of specific cell lineages, both with respect to cell differentiation and analysis of immune effector functions, it also reveals that the nature of the growth factor, i.e., SCF vs. GM-CSF, in context with Hox gene expression can be used to establish progenitor cell lines committed to different lineages.

There is interest in the definition and functional characterization of novel components of Toll-like receptor (TLR) signaling pathways, which control many aspects of inflammation and immune responses. Comparable to many other research areas, however, an important limitation of such research is the availability of suitable cells, particularly for large scale approaches as required for example for proteomic experiments. This is either due to the relative scarcity of specific immune cell types in primary cell cultures, the difficulty of manipulating such primary cells, e.g., by retroviral gene transfer, or the lethal phenotypes of mice deficient for certain genes of interest. The above-described Hoxb8/a9-ERHBD expression system alleviates this problem with respect to granulocytes and monocytes, but does not contribute to the investigation of other cell types, such as dendritic cells (DC) and their subtypes including so-called plasmacytoid DC (pDC), which are major producers of type I interferons (IFNα/β) upon TLR activation and viral infection (Liu (2005) Ann. Rev. Immunol. 23:275-306).

### SUMMARY OF THE INVENTION

The present invention features a method and kits for generating a homogenous population of hematopoietic progenitor cells capable of differentiating into different hematopoietic cell lineages. The method of the invention involves the steps of expressing a hormone-regulated Homeobox (Hox) protein, in a population of bone marrow cells, contacting the bone marrow cells with FMS-like tyrosine kinase 3 ligand (FLT3L) or erythropoietin, and culturing the cells in the presence of hormone, e.g., estrogen, thereby generating a homogenous population of hematopoietic progenitor cells capable of differentiating into a hematopoietic cell lineage such as myeloid, lymphoid, erythropoietic or thrombopoietic cell types. In one embodiment, the Hox protein is expressed via a retrovirus. In another embodiment, the method further includes the step of withdrawing the hormone from the cultured cells to induce differentiation into a hematopoietic cell lineage. In yet another embodiment, the method further includes replacing FLT3L with a differentiation factor such as GM-CSF or M-CSF, or cells expressing a differentiation factor (e.g., OP9 or OP9-DL1) so that the population of hematopoietic progenitor cells is induced to differentiate into different hematopoietic cell lineages including dendritic cells, macrophages, granulocytes, T cells, and B cells.

### DETAILED DESCRIPTION OF THE INVENTION

A method for generating a homogenous population of hematopoietic progenitor cells has now been developed. This method involves delivering an estrogen-regulated Hox gene into largely unfractionated bone marrow (BM) cells, and contacting the cells with a growth factor such as FMS-like tyrosine kinase 3 ligand (FLT3L) or erythropoietin (Epo), to conditionally immortalize an early hematopoietic progenitor cell. Bone marrow cells of use in the instant method can be isolated from any mammalian source, including human, rat, mouse, monkey and the like. Desirably, the bone marrow cells are used without manipulation or fractionation into the various cells types thereof. Bone marrow can be obtained by conventional biopsy from the pelvic bone (iliac crest) or the proximal or distal part of the femur. Moreover, in addition to wild-type cells, the instant bone marrow cells can be obtained from gene-deficient animals.

As used herein, hematopoietic progenitor cells refer to cells that are capable of giving rise to all blood cell lineages. The term “progenitor cell” refers to an undifferentiated cell derived from a stem cell, and is not itself a stem cell. A distinguishing feature of a progenitor cell is that, unlike a stem cell, it does not exhibit self maintenance, and typically is thought to be committed to a particular path of differentiation and will, under appropriate conditions, eventually differentiate along this pathway.

As is known in the art, differentiation is the biological process by which primitive unspecialized cells give rise to progeny having a more specialized function(s). Depending on the Hox gene and growth factor employed, the instant hematopoietic progenitor cells can differentiate into different cell types including myeloid and lymphoid cell types such as macrophages, granulocytes, dendritic cells, and B- and T-lymphocytes, as well as erythropoietic and thrombopoietic cell types such as red blood cells and platelets.

In one embodiment, the BM cells are transduced with Hoxb8 and cultured in the presence of FLT3L to produce cells (referred to herein as Hoxb8-FL cells) capable of differentiating in vitro and in vivo into different myeloid and lymphoid cell types, including macrophages, granulocytes, dendritic cells, and B- and T-lymphocytes. Hoxb8-FL cells do not possess megakaryocyte or erythroid potential, or self-renewal capacity, and correspond functionally and phenotypically largely to so-called lymphoid primed multipotent progenitors (LMPP). Given the simplicity to generate Hoxb8-FL cells from a largely unfractionated BM preparation and the possibility of unlimited proliferative capacity, these cells find application in investigating cell differentiation and immune cell effector functions, as well as in antibody production.

In another embodiment, BM cells are transduced with Hoxa7 and cultured in the presence of Epo to produce cells (referred to herein as Hoxa7-Epo cells) capable of differentiating into different hematopoietic cell lineages, depending on the presence of additional growth factors. For example, removal of hormone and culturing the cells in vitro in the presence of Epo induces generation of (platelet producing) megakaryocytes/erythroid progenitor cells (MkEP), that possess cell lineage potential for both erythropoietic and thrombopoietic cell differentiation, but do not contain myeloid or lymphoid differentiation potential, i.e., they do not generate immune cells. In this respect, Hoxa7-Epo cells can be used in the generation of mature red blood cells and platelets.

Homeobox (Hox) proteins are developmental regulators whose persistent expression has been found to underlie myeloid leukemia, a disease in which the progenitors of macrophages, dendritic cells, and neutrophils are blocked in their differentiation, and can continue to divide as progenitor cells. In addition, exogenous HOXB4 has been shown to enhance stem cell proliferative capacity (U.S. Pat. No. 5,837,507). The instant method takes advantage of the ability of Hox proteins to control cell differentiation thereby immortalizing specific types of progenitor cells. Using a conditional form of Hox, a means of generating unlimited numbers of immature progenitors that can differentiate into myeloid and lymphoid cell types or erythropoietic and thrombopoietic cell types is now provided.

In some embodiments, the Hox gene is Hoxa1, Hoxa2, Hoxa3, Hoxa4, Hoxa5, Hoxa6, Hoxa7, Hoxa9, Hoxa10, Hoxa11, Hoxa13, Hoxb1, Hoxb2, Hoxb3, Hoxb4, Hoxb5, Hoxb6, Hoxb7, Hoxb8, Hoxb9, Hoxb13, Hoxc4, Hoxc5, Hoxc6, Hoxc8, Hoxc9, Hoxc10, Hoxa11, Hoxc12, Hoxc13, Hoxd1, Hoxd3, Hoxd4, Hoxd8, Hoxd9, Hoxd10, Hoxd11, Hoxd12, or Hoxd13. In particular embodiment, the Hox gene encodes Hoxb8. In other embodiments, the Hox gene encodes Hoxa7. Hox genes of mammalian origin are well-known in the art and include, but are not limited to those described in GENBANK Accession Nos. NM_{—}024016 (human Hoxb8 gene encoding Hoxb8 protein of Accession No. NP_{—}076921); NM_{—}001191649 (rat Hoxb8 gene encoding Hoxb8 protein of Accession No. NP_{—}001178578); NM_{—}010461 (mouse Hoxb8 gene encoding Hoxb8 protein of Accession No. NP_{—}034591); NM_{—}006896 (human Hoxa7 gene encoding Hoxa7 protein of Accession No. NP_{—}008827; and NM_{—}010455 (mouse Hoxa7 gene encoding Hoxa7 protein of Accession No. NP_{—}034585).

The function of the Hox protein is made conditional by fusing it, e.g., at its C- or N-terminus, to the hormone-binding domain (hbd) of a steroid receptor. In this respect, the Hox protein is hormone-regulated. A hormone-inducible system allows for high levels of expression, in addition to temporal control of protein activity. In accordance with the instant method, the hbd of a steroid receptor is fused, in frame, with the Hox protein. In the absence of hormone, the fusion protein is held in an inactive state, presumably due to complex formation with hsp90 (Scherrer, et al. (1993) Biochemistry 32:5381-5386). Addition of hormone causes a conformational change that dissociates hsp90, resulting in the rapid activation of the fusion protein (Tsai & O'Malley (1994) Annu. Rev. Biochem. 63:451-486). Hormone binding domains are well-known in the art and include, but are not limited to, the glucocorticoid receptor (GR) ligand binding domain, the estrogen receptor (ER) hbd, the progesterone receptor (PR) hbd, the thyroid hormone receptor (TR) hbd, the mineralocorticoid receptor (MR) hbd and the androgen receptor (AR) hbd. The hbd can be composed of a wild-type sequence that binds its endogenous ligand or, alternatively, the hbd can have a point mutation that mediates binding to a synthetic hormone. For example, a mutant ER hbd has been generated that binds tamoxifen (Vaster, et al. (1996) Oncogene 13:739-748) and a mutant progesterone receptor has been generated that binds RU486 (Kellendonk, et al. (1996) Nucl. Acids Res. 24:1404-1411). In certain embodiments of the present method, the Hox protein is fused to the estrogen receptor hormone binding domain.

Hbds and respective agonistic ligands that can be used to activate the instant Hox protein include the following: ER hbd and estrogen (e.g., β-estradiol), raloxifene, tamoxifen, toremifene, or clomiphene; GR ligand binding domain and dexamethasone; TR hbd and tri-iodothyronine; MR hbd and as aldosterone, deoxycorticosterone and cortisol. In particular embodiments, the hbd and agonistic ligand combination used in the instant method is the ER hbd and estrogen, raloxifene, tamoxifen, toremifene, or clomiphene.

Expression of the hormone-regulated Hox protein in bone marrow cells can be achieved using any conventional recombinant technology for introducing nucleic acids into host cells, including but not limited to, viral transduction, transfection, electroporation, or by a carrier such as an exosome, a liposome, or a functional equivalent thereof. In some embodiments, nucleic acids encoding the hormone-regulated Hox protein are introduced into a cell via a DNA vector (i.e., a naked DNA). In particular embodiments, nucleic acids encoding the hormone-regulated Hox protein are inserted into a viral vector and the virus is used to infect the bone marrow cells.

In some embodiments, the viral vector is a herpes simplex viral vector, an adenoviral vector, or an adeno-associated viral vector (AAV). In other embodiments, the viral vector is a retroviral vector, for example but not limited to, an HIV retroviral vector, a VL 30 vector, a MSCV retroviral vector, or a Harvey Murine Sarcoma Vector. Introduction of the vector into host cells can be carried out by co-culturing the cells with a retroviral producer cell line.

Infected/transfected bone marrow cells are then cultured in tissue culture medium containing a first growth factor and a hormone receptor agonist (to keep the fusion protein active) thereby generating immortalized and undifferentiated hematopoietic progenitor cells. Under these conditions, the cells can be expanded and maintained as a homogenous cell population for at least one, two, three or four months.

As is conventional in the art, a growth factor is a naturally occurring substance capable of stimulating cellular growth, proliferation, and cellular differentiation. In accordance with the instant method the first growth factor is either EPO or FLT3L, and is not stem cell factor, Interleukin (IL)-6, IL-3, IL-5, GM-CSF or Macrophage Colony-Stimulating Factor (M-CSF). Growth factors of the instant method can be introduced into cell cultures as proteins purified from a natural source or as recombinant proteins.

FLT3 has been shown to exhibit a role in DC generation, both in vivo and in in vitro bone marrow cultures (McKenna, et al. (2000) Blood 95:3489-3497; Maraskovsky, et al. (1996) J. Exp. Med. 184:1953-1962; Gilliet, et al. (2002) J. Exp. Med. 195:953-958). Similarly, DC division and homeostatic has been shown to be regulated by FLT3L (Liu & Nussenzweig (2010) Immunol. Rev. 234:45-54). Therefore, when used in combination with expression of Hoxb8, FLT3L is of use in the instant method to generate cells of both myeloid and lymphoid lineage including mature immune cells such as granulocytes, macrophages, dendritic cells, B-cells, T-cells and NK-cells, as well as subtypes of these cells, e.g., osteoclasts.

Erythropoietin is a hormone that regulates red blood cell production. It also has other known biological functions, including participating in the brain's response to neuronal injury (Siren, et al. (2001) Proc. Natl. Acad. Sci. USA 98:4044-4049) and the wound healing process (Haroon, et al. (2003) Am. J. Pathol. 163:993-1000). Epo, along with other cytokines, have been shown to induce lineage specific development of red blood cells from human hematopoietic progenitor cells (U.S. Pat. No. 5,670,351) and erythroid progenitors from bone marrow (U.S. Pat. No. 5,905,041). Therefore, this growth factor alone, or in combination with other growth factors such as thrombopoietin, is of use in inducing erythropoietic and thrombopoietic cell differentiation to generate mature red blood cells and platelets.

Subsequent to expression of the hormone-regulated Hox protein and culture in the presence of hormone and growth factor, populations of immortalized progenitor cells that proliferate indefinitely are generated. Following Hox protein inactivation, cells of myeloid and lymphoid or erythropoietic and thrombopoietic lineage can be produced, depending on the Hox protein and growth factor(s) employed. Accordingly, in one embodiment, the method of the invention further includes the step of withdrawing the hormone from the cultured cells to induce differentiation into a hematopoietic cell lineage. This can be achieved by transferring the cells to medium lacking the hormone or immobilizing the hormone so that it is no longer taken up by the cells. Moreover, cell differentiation can be induced in vitro or in vivo using adoptive mouse transfer models.

As is conventional in the art, the hematopoietic cell lineage includes different myeloid cell types such as dendritic cells, macrophages, osteoclasts, and granulocytes; lymphoid cell types such as B-cells, T-cells, and NK-cells; erythropoietic and thrombopoietic cell types such as red blood cells and platelets. As demonstrated herein, withdrawal or removal of hormone from Hoxb8-FL cells resulted in the differentiation of the HoxB8-FL cells into conventional DC ((cDC) CD11b⁺, CD11c⁺, MHCII⁺, B220⁻) and plasmacytoid DC (CD11b⁻, CD11c⁺, B220⁺) when cultured in the presence of FLT3L. Accordingly, in one embodiment, the step of withdrawing hormone from the cultured cells results in the induction of dendritic cell differentiation in the presence of FLT3L.

As was further demonstrated herein, removal of hormone and replacement of FL3TL with a differentiation factor resulted in the generation of additional cells of the hematopoietic lineage. For example, replacing FLT3L with GM-CSF resulted in the generation of DC (CD11b⁺, CD11c⁺, MHCII⁺, B220⁻) and granulocytes (GR1^{high} CD11c⁻ MHCII⁻), whereas replacing FLT3L with M-CSF provides cells exhibiting the characteristic morphology and marker expression of macrophages (CD11b⁺ MHCII⁻ GR1⁻). Accordingly, in another embodiment of the invention, the growth factor (i.e., FLT3L or EPO) is replaced with a differentiation factor to induce differentiation of the hematopoietic progenitor cells into a cell of the hematopoietic lineage. In a particular embodiment, FLT3L is replaced with GM-CSF to induce DC and granulocyte differentiation. In another embodiment, FLT3L is replaced with M-CSF to induce macrophage differentiation.

In some embodiments, the additional differentiation factor is produced by a population of cells and the hematopoietic progenitor cells are cocultured with the population of cells that produce this additional differentiation factor. In accordance with this embodiment, the population of cells that provide this additional differentiation factor is OP9 cells which induce B cell differentiation. The OP9 cell line was established from newborn op/op mouse calvaria (Nakano, et al. (1994) Science 265:1098-1101) and is readily available under ATCC number CRL-2749. In another embodiment, the population of cells that provide the additional differentiation factor is OP9 cells expressing Delta-Like 1 (DL1) which induce T cell differentiation. OP9-DL1 cells are well known in the art and described by de Pooter & Zúñga-Pflücker ((2007) Curr. Opin. Immunol. 19:163-8).

The instant method finds use in generating cells for pharmacological assays, e.g., high-throughput drug screening, as well as use in basic and medical research and therapeutic applications such as tumor vaccinations or bone marrow reconstitution.

In embodiments pertaining to Hoxb8-FL cells, the instant method and cells are of use in investigating bone marrow homing, cell differentiation (hematopoiesis-specific and general) and immune cell-type specific effector functions. Moreover, it is contemplated that Hoxb8-FL cells could be used in the production of monoclonal antibodies. As Hoxb8-FL cells terminally differentiate, but do not self-renew, adoptive transfer of Hoxb8-FL cells into B-cell deficient mice will lead to generation of one population of mature B-cells. If repetitively challenged with antigen, antigen-specific B-cells are expected to expand and survive, while other B-cells will be lost over time in the absence of bone marrow-derived newly generated B-cells. Such antigen-specific B-cells could then be immortalized by classic cell fusion techniques to establish antibody producing B-cell hybridomas. Such a method would dramatically simplify monoclonal antibody production. In addition, Hoxb8-FL cells from a patient undergoing bone marrow-suppressive treatment, e.g., during bone marrow transplantation, could be established and used therapeutically in case of life-threatening infections.

In comparison to Hoxb8-immortalized cells in the presence of GM-CSF and SCF (Wang, et al. (2006) supra), the instant FLT3L-based system targets an earlier progenitor cell type, which can recapitulate the entire immune cell hematopoiesis, including different myeloid cell types (dendritic cells, macrophages, osteoclasts, granulocytes) and lymphoid cell types (B-cells, T-cells). Moreover, in comparison to Hoxb4- and Nup98-Hox fusions proteins (Antonchuk, et al. (2002) Cell 109:39-45; Ohta, et al. (2007) Exp. Hematol. 35:817-30), which can be used to expand transiently self-renewing hematopoietic stem cells in a mixture of less well-characterized bone marrow cells, the instant method allows stable expansion of a defined population of cells, which can further be manipulated. Both factors, i.e., the possibility to produce essentially unlimited numbers of cells, as well as their homogeneity provides major advantages for basic research applications.

In embodiments pertaining to Hoxa7-Epo cells, the instant method and cells find application in analyzing bone marrow homing, cell differentiation (hematopoiesis-specific and general) and RBC/platelet cell biology. Moreover, Hoxa7-Epo cells could be used to manufacture RBCs and platelets in vitro for blood transfusions. Such cells could be patient-specific or from a ‘common’ and GMP-validated progenitor cell line.

In comparison to Hoxb8-immortalized cells in the presence of GM-CSF and SCF (Wang, et al. (2006) supra), the instant Hoxa7-Epo-based system targets RBC/platelet progenitor cells, which can recapitulate erythropoiesis and thrombopoiesis. The GM-CSF/SCF-system can only be used to generate macrophage- and granulocyte-like cells, respectively. In comparison to Hoxb4- and Nup98-Hox fusions proteins (Antonchuk, et al. (2002) supra; Ohta, et al. (2007) supra), which can be used to expand transiently self-renewing hematopoietic stem cells in a mixture of less well-characterized bone marrow cells, the instant method allows stable expansion of a defined population of cells, which can further be manipulated. This is of particular use in therapeutic applications, such as production and transfusion of in-vitro generated RBC/platelets. Given the uses of cells produced by the instant method, the present invention also features kits. In one embodiment, the kit provides the necessary components for carrying out the method of the invention. In one embodiment, the kit includes a nucleic acid encoding hormone-regulated Hoxb8 protein, i.e., a nucleic acid encoding a hormone-binding domain/Hoxb8 fusion protein, the corresponding hormone that agonizes the hormone-binding domain, and FLT3L ligand or a recombinant cell line expressing the same. Such a kit can further include a differentiation factor such as GM-CSF or M-CSF. Alternatively, the kit can further include a population of OP9 cells or a population of OP9 cells expressing Delta-Like 1.

In another embodiment, the kit includes a nucleic acid encoding hormone-regulated Hoxa7 protein, the corresponding hormone that agonizes the hormone-binding domain, and erythropoietin or a cell line expressing the same.

In still other embodiments, the kit includes a homogenous population of hematopoietic progenitor cells produced by the method described herein. In accordance with this embodiment, the kit can include one or more of the growth factors or differentiation factors and hormone described herein to maintain and differentiate the hematopoietic progenitor cells into a hematopoietic cell lineage.

In yet other embodiments, the kit includes a population of dendritic cells, population of dendritic cells and granulocytes, population of macrophages, population of T cells, or population of B cells generated by the methods described herein.

A kit of the invention can also include all the necessary reagents, such as culture medium and transduction reagents, and vessels or containers as well as step by step instructions.

The invention is described in greater detail by the following non-limiting examples.

### Example 1

### Experimental Procedures

Virus Production.

The plasmids MSCV-ERHBD-Hoxb8 and the ecotropic packaging vector pCL-Eco (Imgenex) were co-transfected into HEK293T cells using LIPOFECTAMINE 2000 (Invitrogen). Eighteen hours after transfection, the supernatant was replaced by medium, followed by cell incubation for 24 hours and collection of virus-containing supernatant. Virus titers were determined on 3T3 MEF cells based on G418 resistance mediated by the retroviral vector.

Generation and Cell Culture of Hoxb8-FL and Hoxb8-SCF Progenitor Cell Lines.

Bone marrow cells were harvested by flushing femurs of 4-8 week old female C57BL/6J or B6/SJL mice with 10 ml RP-10 medium. The cells were pelleted by centrifugation, resuspended in 4 ml RP-10 medium, loaded on 3 ml FICOLL-PAQUE (GE Healthcare) and separated by centrifugation at 450 g for 30 minutes. The entire supernatant was collected (leaving only 500 μl of pellet), diluted with 45 ml PBS/1% FBS, and pelleted for 10 minutes at 800 g. The pellet was resuspended in 10 ml RP-10 medium, centrifuged for 5 minutes at 450 g and resuspended at a concentration of 5×10⁵ cells/ml in RP10 containing IL-3 (10 ng/ml), IL-6 (20 ng/ml) and 1% of cell culture supernatant from a SCF-producing B16 melanoma cell line. After two days of cell culture, cells were collected, resuspended in progenitor outgrowth medium (POM), i.e., RP-supplemented with b-estradiol (1 μM) and either cell culture supernatant from an FLT3L producing B16 melanoma cell line for generation of Hoxb8-FL (5% final concentration) or the described SCF-producing cell line for generation of Hoxb8-SCF (1% final concentration). Cells (2×10⁵) were dispensed in 1 ml per well in a 12-well plate and infected with MSCV vectors (multiplicity of infection 5) by spin inoculation at 450 g for 60 minutes in the presence of LIPOFECTAMINE (Invitrogen). After infection, cells were diluted by adding 1.5 ml POM for 24 hours, followed by removal and replacement of 2 ml of the cell culture medium. During the following cell culture period, cells were dispensed every 3-4 days in fresh medium and transferred into new wells. Once the cell populations were stably expanding, cells were kept at concentrations between 1×10⁵ and 1.5×10⁶ cells/ml medium.

For consequent differentiation experiments, cells were washed twice with PBS containing 1% fetal calf serum (PBS/FBS). For in vitro experiments, cells were resuspended in a concentration of 0.5-2×10⁵ cells/ml in RP-10 containing specific growth factors. For in vivo experiments, cells were resuspended in 0.3 ml PBS/FBS (along with bone marrow cells) and 0.2 ml were transferred into mice via tail vein injection.

Transfer of Hoxb8-FL and BM Cells into Mice.

All mouse studies were carried out in accordance with protocols approved by the Institutional Animal Care and Use Committee at St. Jude Children's Research Hospital. BM-derived DC and BM-derived macrophages were generated by cultivating bone marrow cells in PETRI dishes for six days in medium containing 5% FLT3L-, 2% GM-CSF- or 30% L-cell-conditioned medium derived from factor producing cell lines.

Reagents and Plasmids.

Antibodies used for flow cytometry included those directed to B220, CD3, CD4, CD8, CD45.1, MHCII, CD11b, GR1 (Ly6G), CD11c, IFNα, IL-12p40, αIgM, CD25, CD44, Thy1.2, Sca-1, CD34, FLT3, Ter119, CD19, IgM, AA4.1, CD23, CD21. Antibodies used for stimulation included αCD3, αCD28, and αIgM. ELISA kits for IL-6 and IL-12 were from BD Pharmingen. Griess assays for determination of nitric oxide concentration were as described in the art (Stuehr & Nathan (1989) J. Exp. Med. 169:1543-1555).

Generation of MSCV-Based ERHBD-Hoxb8 Vector.

Mouse Hoxb8 cDNA, which was amplified by PCR using Wehi-3 cDNA as template, was cloned into pMSCVneo (BD Clontech) downstream of a triple hemagglutinin (HA) tag. The estrogen binding domain of the human estrogen receptor (ERHBD) was amplified by overlap extension PCR based on cDNA of the human estrogen receptor 1 (Open Biosystems, clone ID40128594), introducing the characterized G400V mutation, which destabilizes the structure of the hormone binding domain. The mutagenized ERHBD fragment was cloned in frame between triple HA-tag and Hoxb8. The correct sequence of the entire insert was confirmed by DNA sequence analysis.

CpG-DNA (1668) and CpG-DNA (2216) refer to the phosphothioate backbone containing oligonucleotides 1668 (1 μM, TCCATGACGTTCCTGATGCT; SEQ ID NO:1) and 2216 (3 μM, GGGGGACGATCGTCGGGGGG; SEQ ID NO:2) (TIB Molbiol). Other agonists used were LPS (10 ng/ml, E. coli 0127:B8 (Sigma-Aldrich)), IFNγ (10 ng/ml, Peprotech), b-estrogen (Sigma-Aldrich)

Flow Cytometry Analysis.

Single cell suspensions of thymus and spleen were prepared by straining through a 100 μm cell strainer. Peripheral blood was obtained by retro-orbital bleeding and red blood cells were lysed according to established methods. Cells were blocked with antibodies against CD16/CD32 (eBioscience), followed by staining for cell surface markers. For intracellular staining, DC that were first stained for the cell surface markers were fixed with 2% formaldehyde in PBS, followed by incubation with FITC-labelled antibodies against IFNα (or isotype control) and PE-labelled IL-12p40 in PBS containing 0.5% saponin. Flow cytometry analysis was done using a FACSCALIBUR or FACSCANTO II instrument (Becton Dickinson).

### Example 2

### Generation of Hoxb8-FL Cells

The generation of progenitor cell lines of the granulocyte and monocyte lineages using an estrogen-regulated form of Hoxb8 in context of SCF and GM-CSF, respectively, has been described (Wang, et al. (2006) supra). Cell lines established based on Hoxb8 in context with SCF, GM-CSF or FLT3L are referred to herein as Hoxb8-SCF, Hoxb8-GM-CSF or Hoxb8-FL, respectively. To demonstrate that FLT3L could be used to conditionally immortalize a DC precursor, bone marrow cells were briefly expanded in vitro in medium containing IL-3, IL-6 and SCF, and infected with a MSCV-based retrovirus expressing an estrogen-regulated ERHBD-Hoxb8 construct. Following infection, cells were cultured in the presence of estrogen (to activate Hoxb8) and FLT3L. In the absence of Hoxb8 virus, cells did not expand and differentiated into typical DC (Gilliet, et al. (2002) J. Exp. Med. 195:953-58). However, in the presence of activated Hoxb8 and FLT3L, blast-like, stably growing cells expanded with exponential growth characteristics. Growth and survival of these cells strictly depended on FLT3L, and removal of FLT3L led to immediate cell death and cessation of growth. Hoxb8-FL cells could be grown for months in culture without any apparent changes in growth characteristics and phenotype, and also could be subcloned. As such, it was demonstrated that FLT3L could be used to generate Hoxb8-driven, growth factor-dependent cell lines.

### Example 3

### Myeloid Potential of Hoxb8-FL Cells In Vitro

Estrogen removal and hence inactivation of HOXB8 in Hoxb8-SCF and Hoxb8-GM-CSF cells leads to immediate differentiation into granulocytes and macrophages, respectively (Wang, et al. (2006) supra). To characterize the cell fate of Hoxb8-FL cells, estrogen was withdrawn and the growth and phenotype of cells obtained in the presence of FLT3L were analyzed by microscopy and flow cytometry. Primary bone marrow cells were grown and analyzed in parallel under the same conditions in FLT3L-conditioned medium. Hoxb8-FL cells continued to expand until around day six and ceased to grow thereafter but stayed still largely alive until day 8. Phenotypic changes started to appear about three days after estrogen withdrawal, with a decrease in the nucleus to cytoplasm ratio and a slightly increased expression of CD11b and B220 on some cells. In addition, c-Kit, which was found to be highly expressed on non-differentiated Hoxb8-FL cells, was down-regulated in the differentiated cells. Six days after estrogen withdrawal, the cells displayed the typical phenotype of FLT3L-derived DC, i.e., a bi-phenotypic population of so-called conventional DC ((cDC) CD11b⁺, CD11c⁺, MHCII⁺, B220⁻) and plasmacytoid DC (CD11b⁻, CD11c⁺, B220⁺). In contrast to bone marrow-derived DC (BMDC), the Hoxb8-FL-derived cell population was more homogenous and did not contain GR1^{high} CD11c⁻ granulocytes, which are contained in the input population of unfractionated BM and are only gradually lost during the in vitro cell culture. Also, Hoxb8-FL-derived pDC were MHCII negative, while BM-derived pDC showed a more variable, low MHCII expression, a typical phenotype of FLT3L-driven, BM-derived pDC (Liu (2005) supra). This was most likely not due to possible differences in the maturation status of input cells (Hoxb8-FL- or BM cells), as also longer times of differentiation did not lead to up-regulation of MHCII by Hoxb8-FL-derived pDC. However, cell stimulation with known DC activators, such as the TLR9 agonist CpG-DNA, led to strong and homogenous up-regulation of MHCII along with the co-stimulatory molecule CD86 (B7.2) and CD40, all of which are typical markers for DC maturation and critical for instruction of the adaptive immune system, which is a key function of DC (Steinman (2007) Eur. J. Immuno. 37 Suppl 1:S53-60). As such, Hoxb8-FL-derived pDC display a more naïve phenotype than BM-derived pDC in their unperturbed state, but mature efficiently into mature MHCII⁺ DC upon TLR-triggering. The reason for these differences may be due to differences in the cell culture conditions, possibly related to the fact that the vast majority of cells contained in the input BM, i.e., granulocytes and B-cells die during the in vitro cell culture, which may provide cell death-related maturation signals to differentiating cells.

As FLT3 signaling has been shown to be involved in the development of several myeloid lineages (Boiers, et al. (2010) Blood 115:5061-5068), it was determined whether Hoxb8-FL cells might have myeloid potential beyond DC generation. This was achieved by replacing FLT3L, upon estrogen removal, with GM-CSF or M-CSF, which support the in vitro generation of DC and granulocytes, and macrophages, respectively. Even though estrogen withdrawal led to a short period of reduced cell growth and limited cell death, both cytokines supported survival, expansion and eventually the generation of a homogenous, viable population of differentiated cells. Morphologically, cell differentiation became apparent after about three days with changes in cell size, nucleus to cytoplasm ratio and nuclear shape, which was particularly apparent for the development of ‘doughnut cells’, indicative of immature granulocytes. At day six, GM-CSF-driven Hoxb8-FL cells exhibited the classic phenotype of GM-CSF-driven BM cells, characterized by a mixed population of DC (CD11b⁺, CD11c⁺, MHCII⁺, B220⁻) and granulocytes (GR1^{high} CD11c⁻ MHCII⁻). In contrast, M-CSF-cultured cells exhibited the characteristic adherent morphology of macrophages with the typical surface expression of CD11b and lack of MHCII and GR1. Similar to the FLT3L cultures, BM-derived cells were somewhat less homogenous than Hoxb8-FL cells in that they still contained a smaller population of granulocytes, possibly surviving cells from the input BM, where granulocytes (CD11b⁺ GR1^{high}) and B-cells (B220⁺ MHCII⁺) represented the main cell populations. Taken together, Hoxb8-FL cells differentiate in vitro in the presence of specific growth factors into different mature, myeloid cell types, which are phenotypically indistinguishable from their primary BM-derived counterparts. As such, Hoxb8-FL cells possess potential for the major myeloid cell lineages.

### Example 4

### Immune Effector Functions of Hoxb8-FL-Derived Myeloid Cells

As demonstrated herein, differentiated Hoxb8-FL cells were phenotypically indistinguishable from primary BM-derived cells and Hoxb8-FL DC up-regulated MHCII and co-stimulatory molecules comparable to BM DC. To extend these analyses and further establish the functionality of Hoxb8-FL-derived immune cells, selected key immune functions of different Hoxb8-FL- and BM-derived myeloid cell types were analyzed upon TLR activation, including type I interferon (IFNα/β) and IL-12 production by different FLT3L-derived DC subtypes, production of the inflammatory cytokines IL-6 and IL-12 by GM-CSF-driven DC, and nitric oxide (NO) production by M-CSF-driven macrophages, an important part of their first line host defense upon pathogen encounter. As TLR-triggered pDC express IFNα at levels that can be detected by intracellular cytokine staining, FLT3L-driven, Hoxb8-FL-derived B220⁺ pDC were analyzed by flow cytometry. This analysis indicated that B220⁺ cells produced robust levels of IFNα, while IL-12p40 was preferentially expressed by cDC, which are key characteristics of respective cell types (Hemmi, et al. (2003) J. Immunol. 170:3059-64). GM-CSF-driven, Hoxb8-FL-derived DC produced high levels of IL-6 and IL-12 upon CpG-DNA and LPS stimulation, comparable to primary cells. Likewise, M-CSF-driven, Hoxb8-FL-derived macrophages produced NO levels upon treatment with LPS and IFNγ that were virtually indistinguishable from their primary counterparts. As such, based on the analysis of a set of typical immune cell functions, Hoxb8-FL-derived DC and macrophages correspond also in functional terms to BM-derived primary cells.

### Example 5

### Myeloid and Lymphoid Potential of Hoxb8-FL Cells In Vivo

Given the demonstrated multi-myeloid lineage potential of Hoxb8-FL cells and the known activity of FLT3L on very early hematopoietic progenitor cells, including lymphoid-primed multipotent progenitors (LMPP), which have potential for myeloid and lymphoid cell lineages, the lineage potential of Hoxb8-FL cells was determined (Mackarehtschian, et al. (1995) Immunity 3:147-161; Sitnicka, et al. (2002) Immunity 17:463-472; Adolfsson, et al. (2005) supra; McKenna, et al. (2000) supra). To this end, Hoxb8-FL cells, established from CD45.1⁺ B6/SJL mice, were transferred into lethally irradiated CD45.2⁺ C57Bl/6 mice along with a small number of unfractionated CD45.2⁺ BM cells as helper cells and the appearance of mature cell types in the peripheral blood was analyzed over time. In these experiments, Hoxb8-FL cells were also compared with Hoxb8-SCF cells, whose lineage potential has so far only been investigated in in vitro assays (Wang, et al. (2006) supra). Consistent with a largely granulocyte-restricted lineage potential in vitro, Hoxb8-SCF cells generated only CD11b⁺ myeloid cells, most of which were GR1^{high} granulocytes, which was the predominant cell population in the peripheral blood six days after transfer. One week later, Hoxb8-SCF-derived cells were largely lost due to the short half-life of granulocytes and the lack of self-renewal potential of Hoxb8-SCF cells in the absence of exogenous estrogen. In contrast, six days after transfer, Hoxb8-FL cells led to the appearance of a mixed population of CD11b⁺ cells, containing GR1^{high} granulocytes and at least two additional populations with intermediate (GR1^{int.}) and GR1-negative (GR1⁻) phenotype. While cells contained in the GR1^{int.} (and GR1^{high}) populations did not express CD11c, CD11b⁺GR1⁻ cells expressed homogenously high levels of CD11c, which are characteristic phenotypes of CD11b⁺GR1^{int.} blood monocytes and CD11b⁺CD11c⁺GR1⁻ cDC. The latter population was still detectable 14 days after transfer, but declined thereafter and was largely absent at day 28 after transfer. Notably, the transient kinetics of Hoxb8-FL-derived myeloid cells in vivo strongly indicates that Hoxb8-FL cells do not contain self-renewal capacity, but differentiate terminally into mature immune cells whose cell number is determined by the half-life of myeloid cell types in vivo. Intriguingly, in contrast to Hoxb8-SCF cells, which gave rise to exclusively CD11b⁺ myeloid cells, Hoxb8-FL cells generated also B220⁺ CD11b⁻ CD11c⁻ B-lymphocytes, which were the prevalent cell type at day 14 after transfer and persisted for many weeks. Moreover, at day 28 after transfer, a CD3⁺ T-lymphocyte population appeared, which together with Hoxb8-FL-derived B-cells, represented the major cell type at later time points after transfer. A quantitative analysis, based on flow cytometry and peripheral blood cell counts, highlighted the time-dependent appearance of the mature immune cell types in the peripheral blood. Notably, in contrast to myeloid cells and B-cells, Hoxb8-FL-derived T-cells reached only about 10% of physiological T-cell numbers. This phenomenon was not due to the specific experimental protocol used involving irradiation, which is known to influence T-cell maturation (Zlotoff, et al. (2011) Blood 118:1962-1970). Transfer of Hoxb8-FL into non-irradiated IL-7R deficient mice, which were shown to support efficient T-cell development without conditioning irradiation, showed comparably low T-cell reconstitution (Prockop & Petrie (2004) J. Immunol. 173:1604-1611). Thus, in contrast to Hoxb8-SCF cells which have largely granulocyte-restricted lineage potential, Hoxb8-FL cells have myeloid and lymphoid potential which is realized in vivo. The appearance of mature cells in the peripheral blood proceeds in a time-dependent manner with different myeloid cell types emerging already after a few days, followed by B-cells and eventually T-cells, reflecting the well-characterized differences of maturation and survival of respective cell types.

### Example 6

### Characterization of Hoxb8-FL-Derived B- and T-Cells

To characterize Hoxb8-FL-derived B- and T-cells, splenocytes were extracted at later time points after transfer, and phenotype and function were analyzed in more detail. Seven weeks after transfer, splenic B-cells displayed a mature phenotype with a strong bias to follicular B-cells and marginal zone B-cells, and almost complete absence of the different transitional stages of B-cell development. Comparable to BM-derived B-cells, antibody-mediated B-cell receptor crosslinking or TLR9 stimulation induced cell proliferation, a typical consequence of B-cell activation. Hoxb8-FL-derived T-cells were analyzed 5 weeks after transfer and showed an almost identical distribution of CD4- and CD8-single positive cells as BM-derived T-cells. Also, the repertoire of T-cells expressing certain T-cell receptor variable β-chains (TCR Vβ) was highly comparable between Hoxb8-FL- and BM-derived T-cells, as was the proliferative response upon antibody-mediated T-cell receptor crosslinking and CD28 stimulation. As such, Hoxb8-FL cells have lymphoid lineage potential and differentiate in vivo into mature lymphocytes that are phenotypically and functionally comparable to primary BM-derived cells.

### Example 7

### Lymphoid Potential of Hoxb8-FL Cells In Vitro

B- and T-cell development in vivo in adult animals depends on the specific tissue environment provided by BM and thymus, respectively, but can be recapitulated in vitro using stromal cell lines, e.g., OP9 cells and additional exogenous co-factors, i.e., Interleukin (IL)-7 and FLT3L (Lee, et al. (1989) J. Immunol. 142:3875-3883; Hayashi, et al. (1990) J. Exp. Med. 171:1683-1695). For T-cell development, Notch signaling is critical to trigger commitment of early T-cell progenitors to the T-cell lineage, which can be provided by expression of the Notch ligand Delta-like 1 in OP9 cells (OP9-DL1) (Pui, et al. (1999) Immunity 11:299-308; Radtke, et al. (1999) Immunity 10:547-558; Schmitt & Zuniga-Pflucker (2002) Immunity 17:749-756). To determine whether the demonstrated in vivo B- and T-cell potential of Hoxb8-FL cells could be recapitulated in vitro, Hoxb8-FL cells were co-cultured with OP9 or OP9-DL1 cells and their phenotype was analyzed at different time points using surface markers informative for early lymphocyte development, i.e., CD25, CD44, B220 and Thy1. In their non-differentiated state, Hoxb8-FL cells expressed high levels of CD44, but no detectable or very low levels of CD25, B220 and Thy1. Already four days upon co-culture of Hoxb8-FL cells on either OP9 or OP9-DL1 cells, Thy1 was up-regulated, which was transient in the case of OP9-cultured cells and was followed by B220 expression, indicative of pre/pro-B-cell development and commitment to the B-cell lineage (Hardy, et al. (1991) J. Exp. Med. 173:1213-1225). In the case of OP9-DL1 co-cultured cells, Thy1 expression further increased and was sustained during time, accompanied by strong up-regulation of CD25, resulting in the typical phenotype of CD44⁺ CD25⁺ double negative (DN2) T-cell progenitors (Godfrey, et al. (1993) J. Immunol. 150:4244-4252). Under the specific cell culture conditions used, Hoxb8-FL-derived DN2 T-cell progenitors (and BM-derived control cells) sustained this phenotype and did not develop into more mature T-cells. However, when adoptively transferred into lethally irradiated mice, these cells further matured into CD3⁺ CD4⁺/CD8⁺ single positive T-cells, comparable to BM-derived cells. These cells also generated CD11b⁺ myeloid cells, but not B220⁺ B-cells, which is consistent with reports demonstrating the sequential loss of B-cell- and myeloid lineage potential of DN2 and DN3 thymocytes, respectively (Wada, et al. (2008) Nature 452:768-772; Bell & Bhandoola (2008) Nature 452:764-767). Together, Hoxb8-FL cells recapitulate early phases of B- and T-cell development in vitro when provided with appropriate cell culture conditions that support lymphocyte development.

### Example 8

### Hoxb8-FL Cells Lack Megakaryocyte and Erythrocyte Potential

The data described above demonstrate the myeloid and lymphoid potential of Hoxb8-FL cells, but the kinetics of appearance and decline in vivo strongly suggested the lack of self-renewal capacity. As such, it seemed likely that Hoxb8-FL cells either represent a functional equivalent of multipotent progenitor cells (MPP) with full lineage potential including megakaryocyte/erythroid (MkE) potential, or lymphoid primed MPP (LMPP), a cell type defined as committed progenitor with myeloid and lymphoid potential, but loss of MkE potential (Adolfsson, et al. (2005) supra; Yang, et al. (2005) Blood 105:2717-2723). To measure megakaryocyte and erythroid potential, clonal and near-clonal assays, and colony forming unit (CFU)-assays were used, which support megakaryocyte/myeloid cell differentiation and erythroid/myeloid cell differentiation, respectively. LSK cells and unfractionated BM cells (for CFU assays only) were used as positive control. This analysis indicated that LSK cells formed megakaryocytes with the expected frequency, while Hoxb8-FL cells did not show any megakaryocyte potential, but formed exclusively myeloid cells under these conditions. Moreover, under conditions permissive for erythroid development only, i.e., in the presence of erythropoietin (Epo), Hoxb8-FL cells died immediately and did not form any CFU-erythroid (E) or burst forming unit (BFU)-E, while LSK cells gave rise to the expected number of colonies. Under conditions providing additional factors that support myeloid development, i.e., Epo plus SCF, IL-3 and IL-6, Hoxb8-FL cells survived, but formed exclusively CFU-macrophage (M), while LSK cells formed CFU-M in addition to CFU-E and BFU-E. These in vitro data were further confirmed by adoptive transfer of GFP-labeled cells into lethally irradiated mice, where Hoxb8-FL-derived myeloid and lymphoid cell progeny were generated, but neither GFP⁺ red blood cells nor GFP⁺ thrombocytes were detectable at different time points up to four weeks after transfer. As such, Hoxb8-FL cells have myeloid and lymphoid potential, but do not have MkE potential that could be realized under the in vitro or in vivo conditions tested.

### Example 9

### Phenotype Analysis and Homogeneity of Hoxb8-FL Cell Populations

In functional terms, Hoxb8-FL cells match closely a cell type referred to as lymphoid primed MPP (LMPP), which has been shown to have lymphoid and myeloid potential, but lacks MkE potential. These cells were originally characterized based on cell surface markers as FLT3^{hi} LSK cells, and were opposed to FLT3^{-/low} LSK cells with MkE potential (Adolfsson, et al. (2005) supra). To compare Hoxb8-FL cells to LMPPs, flow cytometry analyses were performed using BM-derived LSK cells as control. Comparable to LSK cells, Hoxb8-FL cells were negative for hematopoietic lineage markers, i.e., B220, CD3, CD11b and Ter119. Hoxb8-FL cells also expressed homogenously high levels of c-Kit and FLT3, and also expressed the early hematopoietic progenitor marker CD34. In contrast to LMPP cells, however, Hoxb8-FL cells did not express Sca1.

All Hoxb8-FL cells described were cultured for 4-6 weeks in vitro before analysis, and no significant changes in phenotype or function were observed during extended periods of cell culture, at least for many weeks. These observations resemble experiences of Hoxb8-SCF and Hoxb8-GM-CSF cell lines, and also indicate functional homogeneity of obtained cell populations. To test this aspect, a population of Hoxb8-FL cells was subcloned by single cell sorting and individual clones were expanded, ten of which were investigated in detail with respect to myeloid and lymphoid potential, using described in vitro conditions based on FLT3L- or GM-CSF-conditioned medium and the OP9/OP9-DL1 co-culture system, respectively. This analysis indicated that all ten populations differentiated into expected CD11c⁺ DC, B220⁺ Thy1⁻ CD44⁺ CD25⁻ B-cell- and B220⁻ Thy1⁺ CD44⁺ CD25⁺ T-cell precursors. As such, Hoxb8-FL cells represent a homogenous population of cells, which phenotypically (with the exception of Sca1 expression) and functionally match primary, bone marrow-derived LMPPs.

What is claimed is:

1. A method for generating a homogenous population of hematopoietic progenitor cells capable of differentiating into hematopoietic cell lineages comprising expressing a hormone-regulated Homeobox (Hox) protein in a population of bone marrow cells, contacting the bone marrow cells with FMS-like tyrosine kinase 3 ligand (FLT3L) or erythropoietin, and culturing the cells in the presence of hormone thereby generating a homogenous population of hematopoietic progenitor cells capable of differentiating into different hematopoietic cell lineages.

2. The method of claim 1, wherein the Hox protein comprises Hoxa1, Hoxa2, Hoxa3, Hoxa4, Hoxa5, Hoxa6, Hoxa7, Hoxa9, Hoxa10, Hoxa11, Hoxa13, Hoxb1, Hoxb2, Hoxb3, Hoxb4, Hoxb5, Hoxb6, Hoxb7, Hoxb8, Hoxb9, Hoxb13, Hoxc4, Hoxc5, Hoxc6, Hoxc8, Hoxc9, Hoxc10, Hoxc11, Hoxc12, Hoxc13, Hoxd1, Hoxd3, Hoxd4, Hoxd8, Hoxd9, Hoxd10, Hoxd11, Hoxd12, or Hoxd13.

3. The method of claim 1, wherein the Hox protein is expressed via a retrovirus.

4. The method of claim 1, wherein the hormone is estrogen.

5. The method of claim 1, further comprising the step of withdrawing the hormone from the cultured cells to induce differentiation into a hematopoietic cell lineage.

6. The method of claim 5, wherein the hematopoietic cell lineage comprises megakaryocytes/platelets and red blood cells.

7. The method of claim 5, wherein the hematopoietic cell lineage comprises immune cells of myeloid and lymphoid lineages.

8. The method of claim 5, further comprising replacing FLT3L with a differentiation factor.

9. The method of claim 8, wherein the differentiation factor comprises Granulocyte-Macrophage Colony-Stimulating Factor to induce dendritic cell and granulocyte differentiation.

10. The method of claim 8, wherein the differentiation factor comprises Macrophage Colony-Stimulating Factor to induce macrophage differentiation.

11. The method of claim 8, wherein the differentiation factor is provided by a population of cells.

12. The method of claim 11, wherein the population of cells comprises OP9 cells expressing Delta-Like 1 to induce T cell differentiation.

13. The method of claim 11, wherein the population of cells comprises OP9 cells to induce B cell differentiation.

14. A homogenous population of hematopoietic progenitor cells generated by the method of claim 1.

15. A population of megakaryocytes/erythroid progenitor cells generated by the method of claim 1.

16. A population of dendritic cells generated by the method of claim 7.

17. A population of dendritic cells and granulocytes generated by the method of claim 9.

18. A population of macrophages generated by the method of claim 10.

19. A population of T cells generated by the method of claim 12.

20. A population of B cells generated by the method of claim 13.

21. A kit comprising
(a) a nucleic acid encoding hormone-regulated Homeobox (Hox)-B8 protein,
(b) hormone and
(c) FMS-like tyrosine kinase 3 ligand or a cell line expressing the same.

22. The kit of claim 21, wherein the hormone is estrogen.

23. The kit of claim 21, further comprising a differentiation factor.

24. The kit of claim 23, wherein the differentiation factor comprises Granulocyte-Macrophage Colony-Stimulating Factor.

25. The kit of claim 23, wherein the differentiation factor comprises Macrophage Colony-Stimulating Factor.

26. The kit of claim 21, further comprising a population of OP9 cells expressing Delta-Like 1.

27. The kit of claim 21, further comprising a population of OP9 cells.

28. A kit comprising
(a) a nucleic acid encoding hormone-regulated Homeobox (Hox)-A7 protein,
(b) hormone and
(c) erythropoietin or a cell line expressing the same.

29. The kit of claim 28, wherein the hormone is estrogen.


SEQUENCE LISTING (2 sequences)

SEQ ID NO: 1 - DNA, 20 bp, Artificial sequence
  Synthetic oligonucleotide
tccatgacgttcctgatgct

SEQ ID NO: 2 - DNA, 20 bp, Artificial sequence
  Synthetic oligonucleotide
gggggacgatcgtcgggggg